SEQUENCE LISTING (44 sequences)

SEQ ID NO: 1 - protein, 18 aa, Mus musculus
MEHIQGAWKTISNGFGLK

SEQ ID NO: 2 - protein, 18 aa, Homo sapiens
MEHIQGAWKTISNGFGFK

SEQ ID NO: 3 - protein, 15 aa, Homo sapiens
  MISC_FEATURE (15)..(15): Xaa at position 15 = Asp
HEHKGKKARLDWNTX

SEQ ID NO: 4 - protein, 18 aa, Homo sapiens
HEHKGKKARLDWNTDAAS

SEQ ID NO: 5 - protein, 20 aa, Artificial
  proapoptotic peptide
KMSKDGKKKKKKSXTXCXXX

SEQ ID NO: 6 - protein, 20 aa, Mus musculus
KMSKDGKKKKKKSRTRCTVM

SEQ ID NO: 7 - protein, 20 aa, Homo sapiens
KMSKDGKKKKKKSKTKCVIM

SEQ ID NO: 8 - protein, 30 aa, Artificial
  chimeric peptide
VKKKKIKAEIKIMEHIQGAWKTISNGFGLK

SEQ ID NO: 9 - protein, 30 aa, Artificial
  chimeric peptide
VKKKKIKAEIKIMEHIQGAWKTISNGFGFK

SEQ ID NO: 10 - protein, 30 aa, Artificial
  chimeric peptide
VKKKKIKAEIKIHEHKGKKARLDWNTDAAS

SEQ ID NO: 11 - protein, 32 aa, Artificial
  chimeric peptide
VKKKKIKAEIKIKMSKDGKKKKKKSRTRCTVM

SEQ ID NO: 12 - protein, 32 aa, Artificial
  chimeric peptide
VKKKKIKAEIKIKMSKDGKKKKKKSKTKCVIM

SEQ ID NO: 13 - protein, 14 aa, Artificial Sequence
  cell penetrating peptide
XKKKIKXEIXXXXX

SEQ ID NO: 14 - protein, 12 aa, Artificial
  cell penetrating peptide
VKKKKIKREIKI

SEQ ID NO: 15 - protein, 12 aa, Artificial
  cell penetrating peptide
VKKKKIKAEIKI

SEQ ID NO: 16 - protein, 12 aa, Artificial
  cell penetrating peptide
VKKKKIKKEIKI

SEQ ID NO: 17 - protein, 12 aa, Artificial
  cell penetrating peptide
VKKKKIKNEIKI

SEQ ID NO: 18 - protein, 18 aa, Artificial Sequence
  cell penetrating peptide
RQKRLIRQKRLIRQKRLI

SEQ ID NO: 19 - protein, 18 aa, Artificial Sequence
  cell penetrating peptide
RHSRIGRHSRIGRHSRIG

SEQ ID NO: 20 - protein, 16 aa, Artificial Sequence
  cell penetrating peptide
RHSRIGIIQQRRTRNG

SEQ ID NO: 21 - protein, 16 aa, Artificial Sequence
  cell penetrating peptide
RHSRIGVTRQRRARNG

SEQ ID NO: 22 - protein, 15 aa, Artificial Sequence
  cell penetrating peptide
RRRRRRRSRGRRRTY

SEQ ID NO: 23 - protein, 9 aa, Human immunodeficiency virus
RKKRRQRRR

SEQ ID NO: 24 - protein, 11 aa, Human immunodeficiency virus
YGRKKRRQRRR

SEQ ID NO: 25 - protein, 9 aa, Artificial Sequence
  cell penetrating peptide
RRRRRRRRR

SEQ ID NO: 26 - protein, 11 aa, Artificial Sequence
  cell penetrating peptide
RRRRRRRRRRR

SEQ ID NO: 27 - protein, 32 aa, Artificial Sequence
  cell penetrating peptide
GLFEAIEGFIENGWEGMIDGWYGRRRRRRRRR

SEQ ID NO: 28 - protein, 16 aa, Drosophila
RQIKIWFQNRRMKWKK

SEQ ID NO: 29 - protein, 27 aa, Artificial Sequence
  cell penetrating peptide
GWTLNSAGYLLGKINLKALAALAKKIL

SEQ ID NO: 30 - protein, 33 aa, Maurus Palmatus
GDCLPHLKLCKENKDCCSKKCKRRGTNIEKRCR

SEQ ID NO: 31 - protein, 10 aa, Artificial Sequence
  cell penetrating peptide
KKKKKKKKKK

SEQ ID NO: 32 - protein, 11 aa, Human immunodeficiency virus
YARAAARQARA

SEQ ID NO: 33 - protein, 12 aa, Hepatitis B virus
PLSSIFSRIGDP

SEQ ID NO: 34 - protein, 28 aa, Mus musculus
MANLGYWLLALFVTMWTDVGLCKKRPKP

SEQ ID NO: 35 - protein, 35 aa, Homo sapiens
GGGARKKAAKAARKKAAKAARKKAAKAARKKAAKA

SEQ ID NO: 36 - protein, 19 aa, Mus musculus
LLIILRRRRIRKQAHAHSK

SEQ ID NO: 37 - protein, 23 aa, Homo sapiens
LIRLWSHLIHIWFQNRRLKWKKK

SEQ ID NO: 38 - protein, 22 aa, Avian infectious bursal disease virus
TPWWRLWTKWHHKRRDLPRKPE

SEQ ID NO: 39 - protein, 20 aa, Artificial Sequence
  cell penetrating peptide
GLWRALWRLLRSLWRLLWRA

SEQ ID NO: 40 - protein, 24 aa, Homo sapiens
DSHAKRHHGYKRKFHEKHHSHRGY

SEQ ID NO: 41 - protein, 16 aa, Artificial Sequence
  cell penetrating peptide
KKKEERADLIAYLKKA

SEQ ID NO: 42 - protein, 16 aa, Homo sapiens
  MISC_FEATURE (15)..(15): Xaa at position 15 = Asp
HEHKGKKARLDWNTXX

SEQ ID NO: 43 - protein, 17 aa, Homo sapiens
  MISC_FEATURE (15)..(15): Xaa at position 15 = Asp
HEHKGKKARLDWNTXXX

SEQ ID NO: 44 - protein, 18 aa, Homo sapiens
  MISC_FEATURE (15)..(15): Xaa at position 15 = Asp
HEHKGKKARLDWNTXXXX

(19) United States
(12) Patent Application Publication
(10) Pub. No.: US 2017/0015718 A1
(43) Pub. Date: Jan. 19, 2017

(54) PRO-APOPTOTIC RAS AND RAF PEPTIDES

(71) Applicants: UNIVERSITE PIERRE ET MARIE CURIE (PARIS 6), Paris (FR); INSTITUT CURIE, Paris cedex 05 (FR)

(72) Inventors: ANGELITA REBOLLO GARCIA, PARIS (FR); FARIBA NEMATI, PARIS (FR); DIDIER DECAUDIN, VERRIERES LE BUISSON (FR)

(21) Appl. No.: 14/902,016

(22) PCT Filed: Jul. 3, 2014
(86) PCT No.: PCT/EP2014/064243

§ 371 Date: Dec. 30, 2015

(30) Foreign Application Priority Data
Jul. 3, 2013 (EP) 13305945.1

(51) Int. Cl. C07K 14/47

(52) U.S. Cl. CPC C07K 14/4747; C07K 2319/10; A61K 38/00

(57) ABSTRACT

The invention relates to pro-apoptotic peptides, useful in cancer treatment, and to chimeric peptides comprising a cell penetrating peptide linked to a pro-apoptotic peptide, wherein the pro-apoptotic peptide binds Ras or Raf proteins.

The invention relates to pro-apoptotic peptides, useful in cancer treatment, and to chimeric peptides comprising a cell penetrating peptide linked to a pro-apoptotic peptide, wherein the pro-apoptotic peptide binds Ras or Raf proteins.

### BACKGROUND OF THE INVENTION

The RAS/RAF/MEK/ERK signal transduction pathway regulates cell cycle progression and apoptosis in diverse types of cells. Apoptosis is a genetically programmed cell death and its deregulation is associated among other pathologies, with cancer

Mutations in this pathway are often observed in transformed cell lines and frequently linked with human cancers. The RAS/RAF/MEK/ERK pathway can induce events both associated with cell proliferation and cell cycle arrest.

Ras is a GTPase that regulates multiple cellular processes including cell survival, growth, and differentiation. Following activation via association to GTP, Ras triggers three primary effectors, Raf, PI3K, and Ral-GDS. The three human Ras genes (H, K and N) encode four highly related proteins.

Raf (A, B and C) is a family of three protein serine/threonine kinases that participate in several signalling cascades. These cascades regulate a large variety of processes including apoptosis, cell cycle progression, differentiation, proliferation and transformation to the cancerous state. Raf mutations occur in cutaneous melanomas, thyroid and colorectal cancers. Activation of Raf requires the interaction with Ras-GTP.

Ras mutations occur in 15-30% of all human cancers, and B-Raf mutations occur in 30-60% of melanomas, 30-50% of thyroid cancers, and 5-20% of colorectal cancers (Roskoski et al, 2010).

The RAS-RAF-MEK-ERK pathway has been considered a promising target for anticancer therapy (Maurer et al, 2011, Baines, 2011). B-Raf-inhibitors such as the drug PLX4032 are presently under investigation in clinical trials. However there are also first hints that the tumor cells may develop resistance against such drugs via hyperactivation of N-Ras (Nazarian et al., 2010), which defines a still persisting demand for novel targeted therapeutic approaches.

### SUMMARY OF THE INVENTION

The inventors have mapped binding site of K-Ras to B-Raf and vice-versa. They have then designed peptides showing pro-apoptotic properties, and chimeric peptides wherein a cell penetrating peptide is linked to such pro-apoptotic peptide. The peptides described herein are useful to disturb the Ras/Raf interaction.

The invention thus provides a chimeric peptide construct comprising a cell penetrating peptide linked to a pro-apoptotic peptide, wherein the pro-apoptotic peptide binds a Ras or Raf protein.

The invention further provides such pro-apoptotic peptides, in particular a pro-apoptotic peptide which consists of SEQ ID NO: 1 to 7, and a proteolysis-resistant peptide deriving from said pro-apoptotic peptide by one or more chemical modifications, or a substantially homologous peptide, preferably deriving from SEQ ID NO: 1 to 7 by one or more conservative substitutions.

Another subject of the invention is a nucleic acid that encodes the chimeric peptide construct or the pro-apoptotic peptide as defined herein. A further subject of the invention is a vector comprising said nucleic acid, which is preferably an adenovirus or a lentivirus vector.

The peptides, nucleic acid or vector are useful in treating a tumor.

The chimeric peptide construct, or the pro-apoptotic peptide is useful in treating a tumor in a patient.

In a particular embodiment, the patient is to be administered with a combination of a chimeric peptide construct or pro-apoptotic peptide which binds K-Ras, with a chimeric peptide construct or pro-apoptotic peptide which binds B-Raf.

### LEGENDS TO THE FIGURES

FIGS. 1A and 1B show determination of the binding site of Ras to Raf and vice versa. A) Overlapping dodecapeptides with two amino acid shift covering the whole murine Ras proteins were bound to a solid support. The membrane was incubated sequentially with Raf protein, and anti-Raf antibody, followed by a peroxidase-labeled secondary antibody. The membrane was revealed with ECL system. The sequence corresponding to the identified spots is shown. B) Overlapping dodecapeptides with two amino acids shift covering the Ras binding domain of Raf were synthesized and bound to a solid support. The membrane was incubated with murine Ras protein, followed by anti-Ras antibody and a secondary peroxidase-conjugated antibody. The membrane was revealed using the ECL system. The sequences corresponding to the identified spots are shown.

FIGS. 2A and 2B show in vitro competition of Ras/Raf interaction. Cells were lysed for 20 min at 4° C. in lysis buffer (50 mM Tris (pH 8), 1% Nonidet P-40, 137 mM NaCl, 1 mM MgCl2, 1 mM CaCl2, 10% glycerol, and protease inhibitor mixture). Lysates (800 μg of protein) were immunoprecipitated with the appropriate Ab overnight at 4° C., and protein A-Sepharose was added for 1 h at 4° C. After washing with 1×TBST (20 mM Tris-HCl (pH 7.5), 150 mM NaCl, 0.05% Tween 20), the Ras/Raf interaction was competed using peptides Mut3DPT-Ras (A) or Mut3DPT-Raf+Mut3DPT-Raf2 (B) at a concentration of 1.5 mM for 30 min at room temperature. After a washing, immunoprecipitates were transferred to nitrocellulose and blotted with the corresponding Ab. Membranes were revealed using the ECL system.

FIGS. 3A to 3D show effect of peptides on apoptosis. A) MDA-MB231 cell line (mutated K-Ras) was cultured in the presence of the identified peptides at a concentration of 100 μM for 24 h. Apoptosis was detected by Annexin V-FITC staining and analyzed by flow cytometry. B) MDA-MB231 cell line was cultured as above, but to a concentration of 10 or 25 μM peptides. Apoptosis was estimated by annexin V-FITC staining. C) The K-Ras mutated cell line SW626 was cultured and analyzed as in A. D) The wild type K-RAs cell line BC 227 was cultured and analyzed as in A.

### DETAILED DESCRIPTION OF THE INVENTION

### Definitions

The term “patient” refers to a human or non human animal, preferably a mammal, including male, female, adult and children in need of a treatment wherein a pro-apoptotic effect is desired.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder.

Prophylactic treatment refers to any of: halting the onset, reducing the risk of development, reducing the incidence, delaying the onset, reducing the development, as well as increasing the time to onset of symptoms of a particular disorder.

The term “penetrating peptide” or “cell-penetrating peptide” (or “CPP”) or “shuttle peptide”, as used interchangeably, means that the peptide is able to translocate into cells without causing substantial membrane damage, and can be used as a vector of other molecules when linked to them. The terms refer to cationic cell penetrating peptides, also called transport peptides, carrier peptides, or peptide transduction domains. The CPP, as shown herein, have the capability of inducing cell penetration of a peptide fused to the CPP within 30%, 40%, 50%, 60%, 70%, 80%, 90%, or 100% of cells of a given cell culture population, including all integers in between, and allow macromolecular translocation within multiple tissues in vivo upon systemic administration. A cell-penetrating peptide may also refer to a peptide which, when brought into contact with a cell under appropriate conditions, passes from the external environment in the intracellular environment, including the cytoplasm, organelles such as mitochondria, or the nucleus of the cell, in conditions significantly greater than passive diffusion. This property may be assessed by various methods known by the skilled person. Cell-Penetrating Peptides (CPPs) are also known as protein transduction domains (PTDs), membrane translocating sequences (MTSs), or Trojan peptides.

Two amino acid sequences are “homologous”, “substantially homologous” or “substantially similar” when one or more amino acid residue are replaced by a biologically similar residue or when greater than 80% of the amino acids are identical, or greater than about 90%, preferably greater than about 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs known in the art (BLAST, FASTA, etc.). Preferably, these homologous peptides do not include two cysteine residues, so that cyclization is prevented. Preferably the homologous sequences differ by mutations, such as substitutions, insertions and/or deletions of one or several amino acids. Preferably the homologous sequences differ only by conservative substitution(s).

The term “conservative substitution” as used herein denotes the replacement of an amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties. Examples of conservative substitutions are set out in the Table 1 below:

TABLE 1
Conservative Substitutions I

| SIDE CHAIN CHARACTERISTIC | AMINO ACID |
|---|---|
| Non-polar | G A P I L V |
| Polar-uncharged | C S T M N Q |
| Polar-charged | D E K R |
| Aromatic | H F W Y |
| Other | N Q D E |

Alternatively, conservative amino acids can be grouped as described in Lehninger, 1975, as set out in Table 2, immediately below.

TABLE 2
Conservative Substitutions II

| SIDE CHAIN CHARACTERISTIC | AMINO ACID |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still another alternative, exemplary conservative substitutions are set out in Table 3, immediately below.

TABLE 3
Conservative Substitutions III

| Original Residue | Exemplary Substitution |
|---|---|
| Ala (A) | Val (V), Leu (L), Ile (I) |
| Arg (R) | Lys (K), Gln (Q), Asn (N) |
| Asn (N) | Gln (Q), His (H), Lys (K), Arg (R) |
| Asp (D) | Glu (E) |
| Cys (C) | Ser (S) |
| Gln (Q) | Asn (N) |
| Glu (E) | Asp (D) |
| His (H) | Asn (N), Gln (Q), Lys (K), Arg (R) |
| Ile (I) | Leu (L), Val (V), Met (M), Ala (A), Phe (F) |
| Leu (L) | Ile (I), Val (V), Met (M), Ala (A), Phe (F) |
| Lys (K) | Arg (R), Gln (Q), Asn (N) |
| Met (M) | Leu (L), Phe (F), Ile (I) |
| Phe (F) | Leu (L), Val (V), Ile (I), Ala (A) |
| Pro (P) | Gly (G) |
| Ser (S) | Thr (T) |
| Thr (T) | Ser (S) |
| Trp (W) | Tyr (T) |
| Tyr (Y) | Trp (W), Phe (F), Thr (T), Ser (S) |
| Val (V) | Ile (I), Leu (L), Met (M), Phe (F), Ala (A) |

Pro-Apoptotic Peptide:

The invention makes use of a pro-apoptotic peptide that is a fragment of Ras or Raf protein, or derives therefrom, and binds to Raf or Ras protein, respectively. Preferably the pro-apoptotic peptide is a fragment of human Ras or Raf protein, or derives therefrom. Alternatively, since Ras and Raf proteins are well conserved, fragments originating from other animal species can be used, e.g. mouse or rat Ras or Raf proteins. Most advantageously, the pro-apoptotic peptide binds to human Raf or Ras protein.

According to one embodiment, the pro-apoptotic peptide binds K-Ras.

In a preferred embodiment, the pro-apoptotic peptide comprises or consists of

MEHIQGAWKTISNGFGLK (SEQ ID NO:1) or MEHIQGAWKTISNGFGFK (SEQ ID NO:2);

or a proteolysis-resistant peptide deriving from said pro-apoptotic peptide by one or more chemical modifications, or a substantially homologous peptide, preferably deriving from SEQ ID NO: 1 or 2 by one or more conservative substitutions.

Preferably, amino acids WK and GLK or GFK remain unchanged.

In another embodiment, the pro-apoptotic peptide comprises or consists of HEHKGKKARLDWNTX1 (SEQ ID NO: 3)

wherein X1 is absent, is D or is an amino acid sequence selected from the group consisting of DA, DAA, or DAAS, or

a proteolysis-resistant peptide deriving from said pro-apoptotic peptide by one or more chemical modifications, or a substantially homologous peptide, preferably deriving from SEQ ID NO: 3 by one or more conservative substitutions.

More particularly, the pro-apoptotic peptide may be HEHKGKKARLDWNTDAAS

(SEQ ID NO:4); or a proteolysis-resistant peptide deriving from said pro-apoptotic peptide by one or more chemical modifications, or a substantially homologous peptide, preferably deriving from SEQ ID NO: 4 by one or more conservative substitutions.

Preferably, amino acids K and W remain unchanged.

In another embodiment, the pro-apoptotic peptide binds B-Raf.

The pro-apoptotic peptide then preferably comprises or consists of KMSKDGKKKKKKSX2TX3CX4 (SEQ ID NO:5), wherein X2 and X3 are each independently R or K, X4 is absent or is one to three amino acids;

or a proteolysis-resistant peptide deriving from said pro-apoptotic peptide by one or more chemical modifications, or a substantially homologous peptide, preferably deriving from SEQ ID NO: 5 by one or more conservative substitutions.

More particularly, the pro-apoptotic peptide may be selected from the group consisting of

KMSKDGKKKKKKSRTRCTVM (SEQ ID NO:6);

KMSKDGKKKKKKSKTKCVIM (SEQ ID NO:7); and a proteolysis-resistant peptide deriving from said pro-apoptotic peptide by one or more chemical modifications, or a substantially homologous peptide, preferably deriving from SEQ ID NO: 6 or 7 by one or more conservative substitutions.

Such proteolysis-resistant or homologous peptides induce cell apoptosis, in vitro and/or in vivo. Assays for determining if a molecule, for instance a peptide, induces cell apoptosis are well-known in the art and include, for instance, incubating cells with the candidate peptide and determining if apoptosis is induced by said candidate peptide, e.g. by Annexin V and DAPI or PI labelling of cells and identifying as apoptotic cells, those being Annexin V⁺ and DAPI⁻ or PI⁻. Other methods for determining if a molecule induces cell apoptosis involve following DNA fragmentation by endonucleases, or capsase activations.

Cell Penetrating Peptides:

In preferred embodiments, the pro-apoptotic peptide is linked with at least one cell penetrating peptide, forming a chimeric peptide construct.

Preferably the pro-apoptotic peptide is fused at the C-term of the penetrating peptide.

In a particular embodiment, the pro-apoptotic peptide may be linked to two, three or more penetrating peptides.

Preferably, cell penetrating peptide is a short peptide, of less than about 40 amino acids. Several CPPs are described in http://www.cell-penetrating-peptides.com, or can be designed as described in Gautam et al, 2013, incorporated herein by reference.

Preferably, the cell penetrating peptide comprises or consists of:

a)
(SEQ ID NO: 13)
X₁-KKKIK-Ψ-EI-X₂-X₃

Wherein X₁ is vacant, is a lysine residue, or valine-lysine;

X₂ is vacant, is a lysine residue, or lysine-isoleucine;

X₃ is vacant or is an amino acid sequence of one to 4 amino acids;

and Ψ is any amino-acid;

or a proteolysis-resistant peptide deriving from SEQ ID NO:13 by one or more chemical modifications, or a substantially homologous peptide, especially peptides deriving from SEQ ID NO:13 by one or more conservative substitutions.

b) (RQKRLI)₃ (SEQ ID NO: 18), (RHSRIG)₃ (SEQ ID NO: 19), RHSRIGIIQQRRTRNG (SEQ ID NO: 20), RHSRIGVTRQRRARNG (SEQ ID NO: 21), RRRRRRRSRGRRRTY (SEQ ID NO: 22), or homologous peptides;

c) Tat peptide, polyarginines peptide, HA2-R₉ peptide, Penetratin peptide (Antenna pedia), Transportan peptide, Vectocell® peptide, maurocalcine peptide, decalysine peptide, HIV-Tat derived PTD4 peptide, Hepatitis B virus Translocation Motif (PTM) peptide, mPrP₁₋₂₈ peptide, POD, pVEC, EB1, Rath, CADY, Histatin 5, Antp peptide, Cyt⁸⁶⁻¹⁰¹ peptide.

In an embodiment, in the cell penetrating peptide of a), X3 is vacant, i.e. the cell penetrating peptide is X1-KKKIK-Ψ-EI-X2.

In another embodiment, in the cell penetrating peptide of a), X1 is VK, X2 is KI and X3 is vacant, i.e. the cell penetrating peptide is VKKKKIK-Ψ-EIKI.

Preferably Ψ is arginine, lysine, asparagine, or alanine.

The cell-penetrating peptide can thus be VKKKKIKREIKI (SEQ ID NO:14), VKKKKIKAEIKI (SEQ ID NO:15), VKKKKIKKEIKI (SEQ ID NO:16) or VKKKKIKNEIKI (SEQ ID NO:17).

By “Tat peptide”, it is meant a peptide having the sequence RKKRRQRRR (SEQ ID NO: 23, Tat peptide 2) or YGRKKRRQRRR, (SEQ ID NO: 24).

By “polyarginines peptide”, it is meant a peptide consisting of at least 9 arginines.

Preferably, a polyarginine peptide is a peptide having the sequence R₉ (SEQ ID NO: 25) or R₁₁ (SEQ ID NO: 26).

By “HA2-R₉ peptide”, it is meant a peptide having the sequence GLFEAIEGFIENGWEGMIDGWYG-R₉ (SEQ ID NO: 27).

By “Penetratin peptide”, it is meant a peptide having the sequence RQIKIWFQNRRMKWKK (SEQ ID NO: 28).

By “Transportan peptide” (also called “Antp peptide”), it is meant a peptide having the sequence GWTLNSAGYLLGKINLKALAALAKKIL (SEQ ID NO: 29).

By “Vectocell® peptide”, it is meant a peptide originating from human heparin binding proteins and/or anti-DNA antibodies.

By “Maurocalcine peptide”, it is meant a peptide having the sequence GDCLPHLKLCKENKDCCSKKCKRRGTNIEKRCR (SEQ ID NO: 30).

By “decalysine peptide”, it is meant a peptide having the sequence KKKKKKKKKK (K₁₀) (SEQ ID NO: 31).

By “HIV-Tat derived PTD4 peptide”, it is meant a peptide having the sequence YARAAARQARA (SEQ ID NO: 32).

By “Hepatitis B virus Translocation Motif (PTM) peptide”, it is meant a peptide having the sequence PLSSIFSRIGDP (SEQ ID NO: 33).

By “mPrP₁₋₂₈ peptide”, it is meant a peptide having the sequence MANLGYWLLALFVTMWTDVGLCKKRPKP (SEQ ID NO: 34).

By “POD peptide”, it is meant a peptide having the sequence GGG(ARKKAAKA)₄ (SEQ ID NO: 35).

By “pVEC peptide”, it is meant a peptide having the sequence LLIILRRRRIRKQAHAHSK (SEQ ID NO: 36).

By “EB1 peptide”, it is meant a peptide having the sequence LIRLWSHLIHIWFQNRRLKWKKK (SEQ ID NO: 37).

By “Rath peptide”, it is meant a peptide having the sequence TPWWRLWTKWHHKRRDLPRKPE (SEQ ID NO: 38).

By “CADY peptide”, it is meant a peptide having the sequence GLWRALWRLLRSLWRLLWRA (SEQ ID NO: 39).

By “Histatin 5 peptide”, it is meant a peptide having the sequence DSHAKRHHGYKRKFHEKHHSHRGY (SEQ ID NO: 40).

By “Cyt⁸⁶⁻¹⁰¹ peptide”, it is meant a peptide having the sequence KKKEERADLIAYLKKA (SEQ ID NO: 41).

Chimeric Constructs:

A chimeric peptide construct according to the invention induces cell apoptosis, in vitro and/or in vivo.

The chimeric peptide construct may preferably have a length comprised between 17 to 80 amino acids, preferably between 20 to 70 amino acids, still preferably between 23 to 40 amino acids.

In a preferred embodiment, the chimeric peptide construct is selected from the group consisting of:

(SEQ ID NO: 8)
VKKKKIKAEIKI-MEHIQGAWKTISNGFGLK;
(SEQ ID NO: 9)
VKKKKIKAEIKI-MEHIQGAWKTISNGFGFK;
(SEQ ID NO: 10)
VKKKKIKAEIKI-HEHKGKKARLDWNTDAAS;
(SEQ ID NO: 11)
VKKKKIKAEIKI-KMSKDGKKKKKKSRTRCTVM;
and
(SEQ ID NO: 12)
VKKKKIKAEIKI-KMSKDGKKKKKKSKTKCVIM

or homologous or proteolysis-resistant peptides deriving thereof.

Peptide Preparation:

Peptides described herein can be synthesized using standard synthetic methods known to those skilled in the art, for example chemical synthesis or genetic recombination. In a preferred embodiment, peptides are obtained by stepwise condensation of amino acid residues, either by condensation of a preformed fragment already containing an amino acid sequence in appropriate order, or by condensation of several fragments previously prepared, while protecting the amino acid functional groups except those involved in peptide bond during condensation. In particular, the peptides can be synthesized according to the method originally described by Merrifield.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonyl), Br—Z (2-bromobenzyloyycarbonyl), BzI (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Clz-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Alternatively, the peptide may be synthesized using recombinant techniques. In this case, a nucleic acid and/or a genetic construct comprising or consisting of a nucleotidic sequence encoding a peptide according to the invention, polynucleotides with nucleotidic sequences complementary to one of the above sequences and sequences hybridizing to said polynucleotides under stringent conditions.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

Thus, in another aspect, the invention relates to a host or host cell that expresses (or that under suitable circumstances is capable of expressing) a peptide of the invention; and/or that contains a polynucleotide of the invention or genetic construct of the invention.

The method of producing the peptide may optionally comprise the steps of purifying said peptide, chemically modifying said peptide, and/or formulating said peptide into a pharmaceutical composition.

Further Protection Against Proteolysis:

The N- and C-termini of the peptides described herein may be optionally protected against proteolysis. For instance, the N-terminus may be in the form of an acetyl group, and/or the C-terminus may be in the form of an amide group. Internal modifications of the peptides to be resistant to proteolysis are also envisioned, e.g. wherein at least a —CONH— peptide bond is modified and replaced by a (CH2NH) reduced bond, a (NHCO) retro-inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH-bond.

For instance the peptide may be modified by acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptides of the invention may be composed of amino acid(s) in D configuration, which render the peptides resistant to proteolysis. They may also be stabilized by intramolecular crosslinking, e.g. by modifying at least two amino acid residues with olefinic side chains, preferably C3-C8 alkenyl chains, preferably penten-2-yl chains) followed by chemical crosslinking of the chains, according to the so-called “staple” technology described in Walensky et al, 2004. For instance, amino acids at position i and i+4 to i+7 can be substituted by non-natural aminoacids that show reactive olefinic residues. All these proteolysis-resistant chemically-modified peptides are encompassed in the present invention.

In another aspect of the invention, peptides are covalently bound to a polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide half-life is increased by including the peptide in a biodegradable and biocompatible polymer material for drug delivery system forming microspheres. Polymers and copolymers are, for instance, poly(D,L-lactide-co-glycolide) (PLGA) (as illustrated in US2007/0184015, SoonKap Hahn et al).

Nucleic Acids:

The invention also relates to a polynucleotide comprising or consisting of a nucleotide sequence encoding a peptide according to the invention.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

The genetic constructs of the invention may be DNA or RNA, preferably cDNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises i) at least one nucleic acid of the invention; operably connected to ii) one or more regulatory elements, such as a promoter and optionally a suitable terminator; and optionally also iii) one or more further elements of genetic constructs such as 3′- or 5′-UTR sequences, leader sequences, selection markers, expression markers/reporter genes, and/or elements that may facilitate or increase (the efficiency of) transformation or integration.

In a particular embodiment, the nucleic acid encoding the cell-penetrating peptide of the invention is coupled or fused to a nucleic acid that encodes a peptide or protein of interest. The peptide of interest may be a pro-apoptotic peptide as described herein. More generally it may the peptide or protein of interest may be any peptide or protein to express, such as therapeutic peptide or polypeptide, as well as any antigenic or immunogenic peptide if desired.

The nucleic acid may especially be carried by a viral vector, such as an adenovirus or a lentivirus, for ex vivo or in vivo infection and expression of the chimeric peptide construct or proapoptotic peptide.

Anti-Tumor Therapy:

The peptides as defined herein, or nucleic acids that encode said peptides, are useful in anti-tumor therapy, preferably as adjuvants in combination with an anti-tumor agent, preferably a chemotherapeutic agent.

The anti-tumor therapy of the invention is helpful in eradicating any persistent microscopic malignancy, and/or preventing or delaying relapses.

Furthermore, the peptides (or nucleic acids that encode said peptides) may be used for preventing or treating metastases.

It is thus described a method of treatment of a tumor in a patient in need thereof, which method comprises administering said patient with a proapoptotic peptide of the invention, or a chimeric peptide construct of the invention, or a nucleic acid encoding said construct, preferably in combination with an anti-tumor agent. Anti-tumor agents include chemotherapeutic agents, including inhibitors of DNA replication such as DNA binding agents in particular alkylating or intercalating drugs, antimetabolite agents such as DNA polymerase inhibitors, or topoisomerase I or II inhibitors, or with anti-mitogenic agents such as alkaloids.

The peptides (or nucleic acids that encode said peptides) described herein are useful for the treatment of a tumor, in particular a cancer tumor, preferably in a human patient. The tumor may be cancer, such as a haematologic cancer, in particular acute myelogenous leukaemia (AML), chronic lymphocytic leukaemia (CLL), multiple myeloma, Hodgkin's disease, non-Hodgkin's lymphoma, B cell lymphoma, cutaneous T cell lymphoma, or a non-haematologic cancer, for instance brain, epidermoid (in particular lung, breast, ovarian), head and neck (squamous cell), bladder, gastric, pancreatic, head, neck, renal, prostate, colorectal, oesophageal or thyroid cancer, and melanoma.

Different types of cancers may include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, lymphoma, leukemia, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma, uveal melanoma and breast cancer.

More particularly the peptides described herein (or nucleic acids that encode said peptides) are useful in the treatment of cancers which exhibit a mutation of a Ras or Raf gene.

In a preferred embodiment, the cancer tumor is a colorectal cancer or a melanoma.

Pharmaceutical Compositions:

The peptides of the invention (or nucleic acid that encodes said peptide) may be administered by any convenient route including intravenous, oral, transdermal, subcutaneous, mucosal, intramuscular, intrapulmonary, intranasal, parenteral, rectal, vaginal and topical. Intranasal route is of particular interest.

Advantageously, intra-tumoral administration is also contemplated.

The peptides (or nucleic acid that encodes said peptide) are formulated in association with a pharmaceutically acceptable carrier.

The pharmaceutical composition may also include any other active principle, such as in particular an anti-tumor agent, such as those described above.

In a preferred embodiment, the peptides (or nucleic acid that encodes said peptide) may be administered by electroporation. Electroporation, also known as electropermeabilization or electroinjection, is the permeabilization of cell membranes as a consequence of the application of certain short and intense electric fields across the cell membrane, the cells or the tissues. Typically, electroporation consists of injecting compounds, preferably via intramuscular or intradermal route, followed by applying a series of electric pulses by means of electrodes connected to a generator. The conditions for applying an electric field in the injection zone are now well known to those persons skilled in the art, and are in particular described in the U.S. Pat. No. 5,468,223. Those persons skilled in the art will be able to adapt these conditions according to each case. The electric field may be 50-200 microseconds pulses of high-strength electric fields in the range of 1-5000 V/cm and with a frequency between 0.1 and 1,000 hertz. Typically, a sequence of eight 100 microseconds pulses of 1000-1500 V/cm with a frequency of 1 hertz is applied.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product.

The dosing is selected by the skilled person so that a pro-apoptotic effect is achieved, and depends on the route of administration and the dosage form that is used. Total daily dose of peptides (or nucleic acid that encodes said peptide) administered to a subject in single or divided doses may be in amounts, for example, of from about 0.001 to about 100 mg/kg body weight daily and preferably 0.01 to 10 mg/kg/day. A daily dosage of about 5 mg/kg is preferred. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

Preferably the peptide construct (or nucleic acid that encodes said peptide) is administered once a day during a period of at least one week, preferably at least two weeks.

Combinations:

In a particular embodiment, the patient is to be administered with a combination of a chimeric peptide construct or pro-apoptotic peptide which binds K-Ras, with a chimeric peptide construct or pro-apoptotic peptide which binds B-Raf. Simultaneous administration (i.e., at the same time, as a single composition or separate compositions), or sequential administration is encompassed.

The invention further provides a pharmaceutical composition comprising a chimeric peptide construct or pro-apoptotic peptide which binds K-Ras, in combination with a chimeric peptide construct or pro-apoptotic peptide which binds B-Raf.

It is also provided a kit comprising a container containing a chimeric peptide construct or pro-apoptotic peptide which binds K-Ras, and a container containing a chimeric peptide construct or pro-apoptotic peptide which binds B-Raf.

Further aspects and advantages of the present invention will be disclosed in the following experimental section, which should be regarded as illustrative and not limiting the scope of the present application.

### EXAMPLES

### Example 1

### Identification of Binding Site of K-Ras to B-Raf and Vice Versa

1.1. Materials and Methods

Peptide Synthesis and Sequence

Peptides were synthesized in an automated multiple peptide synthesizer with solid phase procedure and standard Fmoc chemistry. The purity and composition of the peptides were confirmed by reverse phase HPLC and by amino acid analysis. These peptides were used for protein-protein interaction competition studies or cell culture.

Ras/Raf binding assay on cellulose-bound peptides containing Ras or Raf sequences

Overlapping peptides covering the whole K-Ras or Ras Binding Domain (RBD) of B-Raf were prepared by automated spot synthesis into an amino-derivatized cellulose membrane as previously described (Frank and Overwin, 1996; Gausepohl, et al, 1992). The membranes were blocked, incubated with purified K-Ras or B-Raf protein and, after several washing steps, incubated with anti-Ras or anti-Raf Ab followed by the PO-conjugated secondary Ab. Protein interactions were visualized using the ECL system.

1.2. Results

Identification of Binding Site of K-Ras to B-Raf and Vice Versa

To identify peptides containing K-Ras sequence able to bind to Raf, the whole sequence of K-Ras was synthetized as series of dodecapeptides that were bound to a nitrocellulose support.

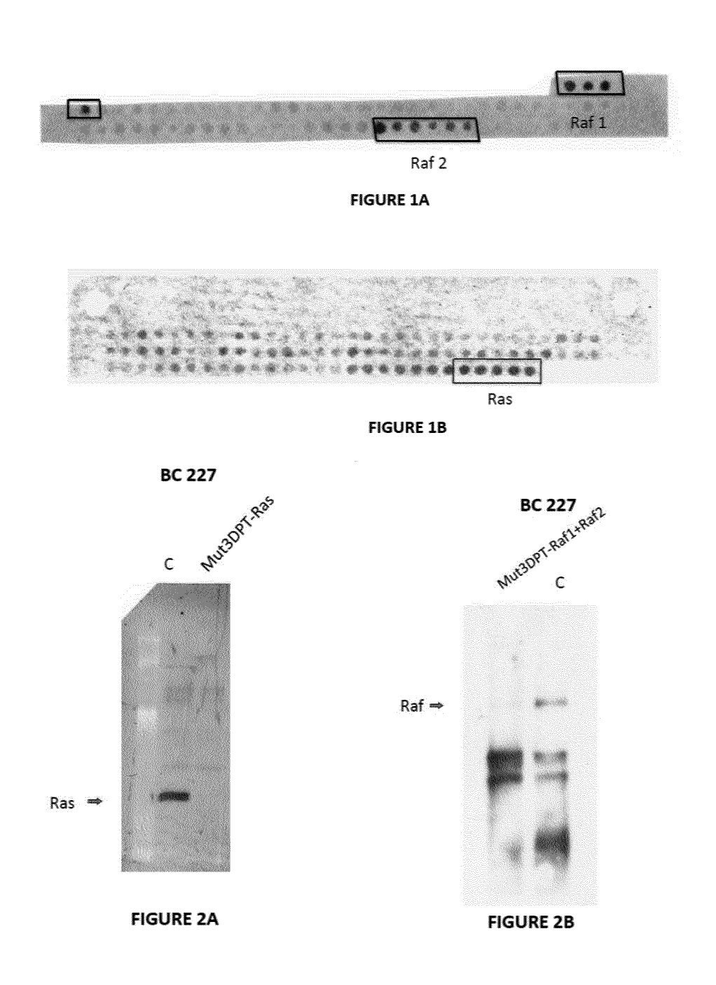

The inventors identified two overlapping sequences, one of four dodecapeptides and the other one of six dodecapeptides, corresponding to Raf 1 and Raf2, respectively. We have identified a sequence of 20 amino acids that corresponds to the binding site of Raf to K-Ras (FIG. 1A). The two sequences are: Raf1 MEHIQGAWKTISNGFGLK and Raf2 HEHKGKKARLDWNTDAAS.

Similarly, to identify peptides containing B-Raf sequence able to bind to K-Ras, the Ras binding domain (RBD) of B-Raf was synthesized as series of dodecapeptides that were bound to a solid support. The inventors have hence identified the Ras binding domain of Raf (FIG. 1B). The sequence is: KMSKDGKKKKKKSRTRCTVM.

### Example 2

### Design and Characterization of Mut3DPT-Ras, Mut3-DPT-Raf1 and Mut3DPT-Raf2

2.1. Materials and Methods

Cells

Human K-Ras mutated cell line MDA-MB231 and SW646, as well as the K-Ras wt

BC52 cell line, isolated from primary human cancer xenografts were cultured in DMEM and RPMI medium respectively supplemented with 10% of FCS.

In Vitro Competition of Ras/Raf Interaction

The Ras/Raf interaction was competed using peptides corresponding to the binding site of Ras to Raf and vice versa. Lysates from MDA-MB321 or BC52 cell lines were immunoprecipitated with anti-Ras or anti-Raf Ab, and protein A-Sepharose was added. The Ras/Raf interaction was competed with 1.5 mM of peptides (30 min, room temperature). After washing steps, immunoprecipitates were transferred to nitrocellulose and blotted with the corresponding Ab. Proteins were detected using the ECL system.

2.2. Results

The inventors chemically synthesized the three cell penetrating peptides composed of a shuttle, Mut3DPT-Sh1 (VKKKKIKAEIKI) associated to the binding site of K-Ras to Raf and vice versa. The peptides are:

Mut3 DPT-Ras
(SEQ ID NO: 12)
VKKKKIKAEIKIKMSKDGKKKKKKSKTKCVIM
Mut3 DPT-Raf1
(SEQ ID NO: 9)
VKKKKIKAEIKIMEHIQGAWKTISNGFGFK
Mut3 DPT-RAf2
(SEQ ID NO: 10)
VKKKKIKAEIKIHEHKGKKARLDWNTDAAS

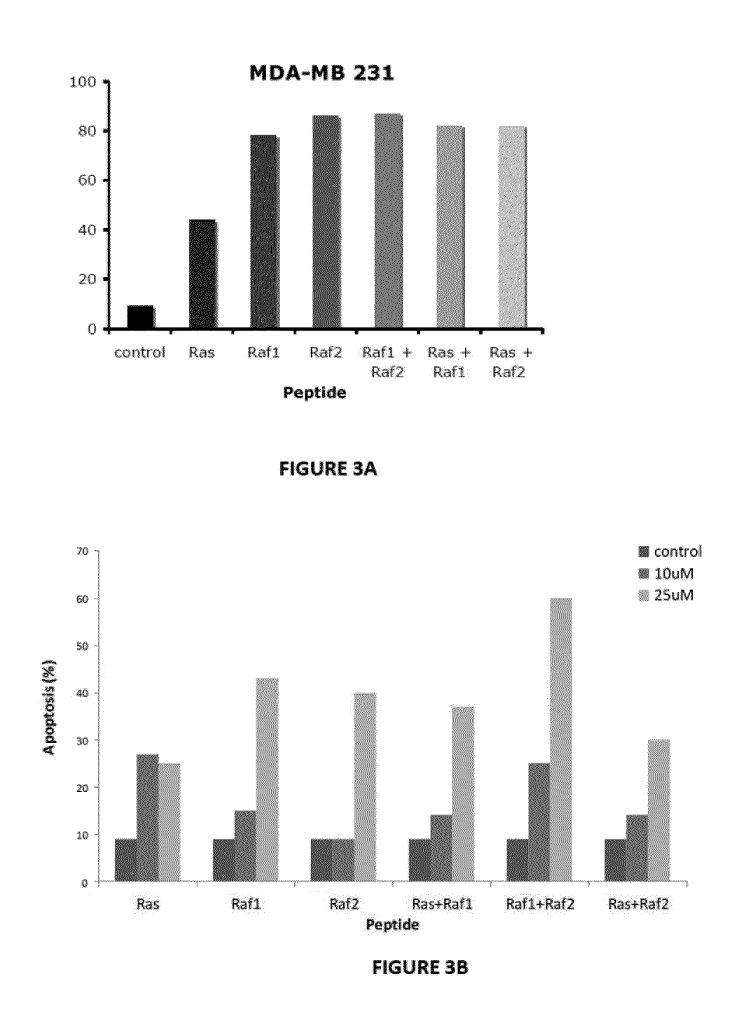

To determine the capacity of the peptides Mut3DPT-Ras (SEQ ID NO: 12), Mut3DPT-Raf1 (SEQ ID NO:9) and Mut3DPT-Raf2 (SEQ ID NO:10) to target the Ras/Raf interaction, the inventors performed in vitro competition assays. As illustrated in FIGS. 2A and 2B, the inventors were able to compete in vitro the interaction Ras/Raf using either Ras or Raf1+Raf2 peptides.

### Example 3

### Effect of Mut3DPT-Ras, Mut3-DPT-Raft and Mut3DPT-Raf2 Peptides on Apoptosis

3.1. Materials and Methods

Detection of Apoptosis by Annexin-V-FITC Staining

Apoptotic cells were detected using Annexin-V (-FITC from BD biosciences) as described by the manufacturer. Briefly, the cells were washed in 1× binding buffer, centrifugated and then resuspended in 200 μl of 1× binding buffer containing Annexin V-FITC (0.1 μg/ml) and PI (0.5 μg/ml). After incubation at room temperature in the dark for 10 min, cells were analyzed by flow cytometry. Data acquired by FACSCalibur (BD biosciences) were analyzed with Cellquest Pro software.

3.2. Results

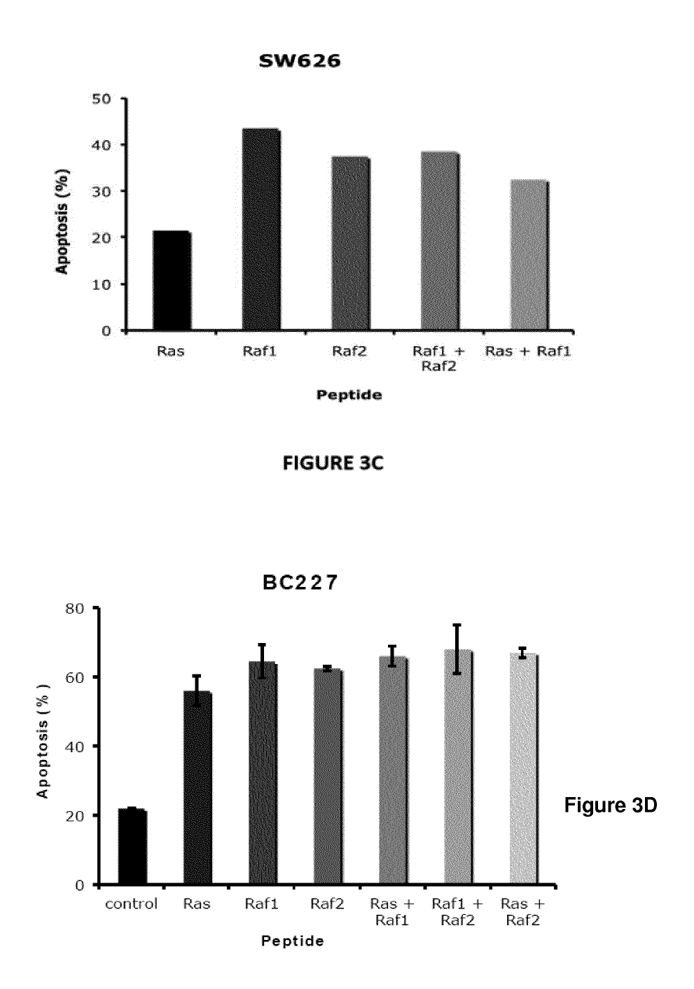

The inventors analyzed the capacity of these peptides to induce apoptosis in K-Ras mutated and wild type cell lines. As illustrated on FIG. 3A, all the peptides were able to induce apoptosis on the K-Ras mutated cell line MDA-MB231.

The inventor further analyzed whether using lower doses of peptides, they could be able to detect an additive effect using the peptide to a concentration of 25 μM for 24 h. FIG. 3B shows that addition to the culture medium of both Raf1 and Raf 2 peptides has an additive effect, shown higher level of apoptosis suggesting that blocking of both sites should be necessary for a complete effect.

The effect of the peptides was also analyzed in other K-Ras mutated cell line, SW626. FIG. 3C shows that all the peptides have apoptotic effect. As in the previous figure, the Ras peptide induced lower level of apoptosis, relative to control non treated cells when the peptides were used at 100 μM for 24 h.

Finally, the peptides were tested in a K-Ras wild type cell line, BC227 (FIG. 3D), showing a strong apoptotic effect when used at a concentration of 100 μM for 24 h.

The new designed peptides, Mut3DPT-Ras, Mut3DPT-Raf1 and Mut3DPT-Raf2 have a potential therapeutic effect, as illustrated by their apoptotic potential.

These peptides have further been tested for their ability to induce apoptosis of several colon and lung cancer cell lines, as well as primary tumor cells of chronic lymphocytic leukemia (CLL). An apoptotic effect was observed. Noteworthy Mut3DPT-Raf2 was shown to cause apoptosis of colon cancer cell line HCT116 at a very low concentration (12.5 μM for 72 h). Mut3DPT-Ras showed a marked effect on CLL B and non-B cells.

### REFERENCES

Baines A T. 2011. Future Med Chem. 3:1787;
Frank, R., H. Overwin. 1996. Methods Mol. Biol. 66: 149-144;
Gausepohl, et al. 1992. Pept. Res. 5: 315-314;
Gautam et al, Journal of Translational Medicine 2013, 11:74;
Lehninger, (1975) Biochemistry, Second Edition, Worth Publishers, Inc. New-York: N.Y., pp. 71-77;
Marangoni et al., 2007. Clinical Cancer REsearch 13, pages 3989-3998;
Maurer G, Tarlkowski B and Baccarini M. 2011. Oncogene 30:3477;
Nazarian et al, 2010 Nature. 468(7326):973-7;
Roskoski R, 2010. Biochem Biophys Res Commun.; 399(3):313-7;
Walensky et al, 2004, Science 305:1466-1470.

1-21. (canceled)

22. A chimeric peptide construct comprising a cell penetrating peptide linked to a pro-apoptotic peptide, wherein the pro-apoptotic peptide binds a Ras or Raf protein.

23. The chimeric peptide construct of claim 22, wherein the pro-apoptotic peptide binds K-Ras.

24. The chimeric peptide construct of claim 23, wherein the pro-apoptotic peptide comprises or consists of:
(SEQ ID NO: 1)
MEHIQGAWKTISNGFGLK
or
(SEQ ID NO: 2)
MEHIQGAWKTISNGFGFK;
or
a proteolysis-resistant peptide derived from said pro-apoptotic peptide by one or more chemical modifications, or a substantially homologous peptide derived from SEQ ID NO: 1 or 2 by one or more conservative amino acid substitutions.

25. The chimeric peptide construct of claim 23, wherein the pro-apoptotic peptide comprises or consists of:
HEHKGKKARLDWNTX₁ (SEQ ID NO:3) wherein X₁ is absent, is D or is an amino acid sequence selected from the group consisting of DA, DAA, or DAAS;
a proteolysis-resistant peptide derived from said pro-apoptotic peptide by one or more chemical modifications, or a substantially homologous peptide derived from SEQ ID NO:3 by one or more conservative amino acid substitutions; or
HEHKGKKARLDWNTDAAS (SEQ ID NO:4), or a proteolysis-resistant peptide derived from said pro-apoptotic peptide by one or more chemical modifications, or a substantially homologous peptide derived from SEQ ID NO: 4 by one or more conservative amino acid substitutions.

26. The chimeric peptide construct of claim 22, wherein the pro-apoptotic peptide binds B-Raf.

27. The chimeric peptide construct of claim 26, wherein the pro-apoptotic peptide comprises or consists of:
KMSKDGKKKKKKSX₂TX₃CX₄ (SEQ ID NO:5) wherein X₂ and X₃ are each independently R or K, X₄ is absent or is one to three amino acids;
a proteolysis-resistant peptide derived from said pro-apoptotic peptide by one or more chemical modifications, or a substantially homologous peptide derived from SEQ ID NO: 5 by one or more conservative amino acid substitutions;
a pro-apoptotic peptide is selected from the group consisting of:
(SEQ ID NO: 6)
KMSKDGKKKKKKSRTRCTVM;
(SEQ ID NO: 7)
KMSKDGKKKKKKSKTKCVIM;
or
a proteolysis-resistant peptide derived from said pro-apoptotic peptide by one or more chemical modifications, or a substantially homologous peptide derived from SEQ ID NO: 6 or 7 by one or more conservative amino acid substitutions.

28. The chimeric peptide construct of claim 22, wherein said cell-penetrating peptide is selected from:
a)
(SEQ ID NO: 13)
X₁-KKKIK-Ψ-EI-X₂-X₃;
wherein X₁ is vacant, is a lysine residue, or valine-lysine;
X₂ is vacant, is a lysine residue, or lysine-isoleucine;
X₃ is vacant or is an amino acid sequence of one to 4 amino acids;
and Ψ is any amino-acid;
a proteolysis-resistant peptide derived from SEQ ID NO: 13 by one or more chemical modifications or a substantially homologous peptide derived from SEQ ID NO:13 and containing one or more conservative amino acid substitutions;
b)
(SEQ ID NO: 18)
(RQKRLI)₃,
(SEQ ID NO: 19)
(RHSRIG)₃,
(SEQ ID NO: 20)
RHSRIGIIQQRRTRNG,
(SEQ ID NO: 21)
RHSRIGVTRQRRARNG,
(SEQ ID NO: 22)
RRRRRRRSRGRRRTY;
or
c) Tat peptide, polyarginines peptide, HA2-R₉ peptide, Penetratin peptide, Transportan peptide, Vectocell peptide, maurocalcine peptide, decalysine peptide, HIV-Tat derived PTD4 peptide, Hepatitis B virus Translocation Motif (PTM) peptide, mPrP₁₋₂₈ peptide, POD, pVEC, EB1, Rath, CADY, Histatin 5, Antp peptide, or Cyt⁸⁶⁻¹⁰¹ peptide.

29. The chimeric peptide construct of claim 28, wherein said cell-penetrating peptide is:
a)
(SEQ ID NO: 13)
X₁-KKKIK-Ψ-EI-X₂-X₃;
wherein Ψ is arginine, alanine, lysine, or asparagines; and
wherein said cell-penetrating peptide is:
a)
(SEQ ID NO: 13)
X₁-KKKIK-Ψ-EI-X₂-X₃;
wherein Ψ is arginine, alanine, lysine, or asparagine; and
X₁ is valine-lysine;
X₂ is lysine-isoleucine; and
X₃ is vacant.

30. The chimeric peptide construct of claim 29, wherein said cell-penetrating peptide is:
VKKKKIKREIKI (SEQ ID NO:14), VKKKKIKAEIKI (SEQ ID NO:15), VKKKKIKKEIKI (SEQ ID NO:16) or VKKKKIKNEIKI (SEQ ID NO:17), and wherein the chimeric peptide construct is selected from the group consisting of:
(SEQ ID NO: 8)
VKKKKIKAEIKI-MEHIQGAWKTISNGFGLK;
(SEQ ID NO: 9)
VKKKKIKAEIKI-MEHIQGAWKTISNGFGFK;
(SEQ ID NO: 10)
VKKKKIKAEIKI-HEHKGKKARLDWNTDAAS;
(SEQ ID NO: 11)
VKKKKIKAEIKI-KMSKDGKKKKKKSRTRCTVM;
and
(SEQ ID NO: 12)
VKKKKIKAEIKI-KMSKDGKKKKKKSKTKCVIM

31. A method for treating a tumor in a patient, which method comprises administering to said patient the chimeric peptide construct of claim 22.

32. The method of claim 31, wherein the patient is administered with a combination of a chimeric peptide construct which binds K-Ras and a chimeric peptide construct which binds B-Raf.

33. The method of claim 31, wherein the tumor is a haematologic cancer, acute myelogenous leukaemia (AML), chronic lymphocytic leukaemia (CLL), multiple myeloma, Hodgkin's disease, non-Hodgkin's lymphoma, B cell lymphoma, cutaneous T cell lymphoma, a non-haematologic cancer, brain cancer, epidermoid cancer, lung cancer, breast cancer, ovarian cancer, head and neck cancer, bladder cancer, gastric cancer, pancreatic cancer, head cancer, neck cancer, renal cancer, prostate cancer, colorectal cancer, oesophageal cancer or thyroid cancer, and melanoma.

34. A nucleic acid that encodes the chimeric peptide construct as defined in claim 22.

35. A vector comprising the nucleic acid of claim 34.

36. A method for treating a tumor, which comprises administering the nucleic acid of claim 34.

37. A method for treating a tumor in a patient, which method comprises administering to said patient a pro-apoptotic peptide which consists of any of SEQ ID NO: 1 to 7, and a proteolysis-resistant peptide derived from said pro-apoptotic peptide by one or more chemical modifications, or a substantially homologous peptide derived from any of SEQ ID NO: 1 to 7 by one or more conservative amino acid substitutions.

38. The method of claim 37, wherein the tumor is a haematologic cancer, acute myelogenous leukaemia (AML), chronic lymphocytic leukaemia (CLL), multiple myeloma, Hodgkin's disease, non-Hodgkin's lymphoma, B cell lymphoma, cutaneous T cell lymphoma, a non-haematologic cancer, brain cancer, epidermoid cancer, lung cancer, breast cancer, ovarian cancer, head and neck cancer, bladder cancer, gastric cancer, pancreatic cancer, head cancer, neck cancer, renal cancer, prostate cancer, colorectal cancer, oesophageal cancer or thyroid cancer, and melanoma.

39. A pro-apoptotic peptide which consists of any of SEQ ID NO: 1 to 4, and a proteolysis-resistant peptide derived from said pro-apoptotic peptide by one or more chemical modifications, or a substantially homologous peptide derived from any of SEQ ID NO: 1 to 4 by one or more conservative amino acid substitutions.

40. A nucleic acid that encodes the pro-apoptotic peptide construct as defined in claim 39.

41. A vector comprising the nucleic acid of claim 40.

42. A method for treating a tumor in a patient, which method comprises administering to said patient a nucleic acid that encodes a pro-apoptotic peptide which consists of any of SEQ ID NO: 1 to 7, or a proteolysis-resistant peptide derived from said pro-apoptotic peptide by one or more chemical modifications, or a substantially homologous peptide, derived from any of SEQ ID NO: 1 to 7 by one or more conservative amino acid substitutions.
